(19) United States
(12) Patent Application Publication
(10) Pub. No.: US 2017/0209649 A1
(43) Pub. Date: Jul. 27, 2017

(54) Syringe Injector

(71) Applicant: Jason Wei, Arcadia, CA (US)

(72) Inventor: Jason Wei, Arcadia, CA (US)

(21) Appl. No.: 15/002,925

(22) Filed: Jan. 21, 2016

(51) Int. Cl. A61M 5/315; A61M 5/20

(52) U.S. Cl. CPC A61M 5/31515; A61M 5/2033; A61M 5/204; A61M 5/31576; A61M 5/31583; A61M 2205/586; A61M 2005/2026; A61M 2005/3152

(57) ABSTRACT

A reciprocating syringe injector includes an inside arm and an outside arm pivotally mounted to the inside arm. A spring bias biases the inside arm away from the outside arm. A small syringe retainer is formed on the inside arm. A ratchet member extends from the inside arm toward the outside arm. The ratchet member has ratchet teeth protruding from the ratchet member. A ratchet engagement extension is pivotally mounted to the outside arm. The ratchet engagement extension has a ratchet engagement edge that engages the ratchet teeth formed on the ratchet member. The reciprocating syringe injector also optionally includes a large reservoir retainer mounted to the small syringe retainer. The large reservoir retainer could also be mounted anywhere on the inside arm.

### FIELD OF THE INVENTION

The present invention is in the field of reciprocating syringe injectors.

### DISCUSSION OF RELATED ART

Syringes are used for injecting or withdrawing fluids or liquids. However, repetitive manual operation of syringes can lead to strain. For example, physicians use syringes for medical procedures such as neural prolotherapy (NPT), which involves using a small gauge needle of 27 or 30 gauge to inject a sugar & bicarbonate solution into subcutaneous tissue to calm nerves. The procedure typically requires 100 cc of solution and doctors typically use 10 cc syringes. Unfortunately, small gauge needles can lead to stress on the thumb joints, joint tissues or osteoarthritis from the stress. Therefore, a variety of different have been invented to reduce risk of repetitive strain injuries.

U.S. Pat. No. 2,722,215 entitled Spring-Actuated Medical Syringe, dated Nov. 1, 1955 by Stig-Ake Dahlgren, the disclosure of which is incorporated herein by reference, presents a new and improved syringe with an injection needle that may be retracted and propelled by a spring upon release of a trigger. The Dahlgren invention is intended to improve the difficulty associated with injecting a needle into a user's own body or into others. U.S. Pat. No. D249,808 dated Oct. 3, 1978, entitled Syringe Pumping Handle by William G. Bloom and Gerald W. Schmidt, the disclosure of which is incorporated herein by reference, presents a different invention for simplification of syringe use. Rather than using a spring to decrease strain on the user, the ornamental design presented in this patent provides a handle that reduces the force needed to dispense fluid from the syringe.

In U.S. Pat. No. 6,616,634 dated Sep. 9, 2003, entitled Ergonomic Syringe by Phillip David Benz et al., the disclosure of which is incorporated herein by reference, the large handgrip and locations of the finger grips on the ergonomic syringe increase comfort and reduce the strain and risk of injury associated with injection using a syringe. U.S. Pat. No. D576,273 dated Sep. 2, 2008, entitled Hand Held Manual Ergonomic Syringe Dispenser Pistol by Scott McClintok et al., the disclosure of which is incorporated herein by reference, provides a different design for a hand held manual ergonomic syringe dispenser pistol. The ornamental design presented in this patent is also intended to facilitate comfortable and safe use of a syringe.

United States Patent Application Publication Number US 2010/0249719 dated Sep. 30, 2010, entitled Methods of Manually Injecting/Aspirating Fluids Through Small Diameter Catheters and Needles and Manual Injection/Aspiration Systems Including Small Diameter Catheters and Needles, by Shawn P. Fojtik, the disclosure of which is incorporated herein by reference, presents a similar invention with a different purpose. The system presented provides a scissor-grip handle that enables use of a single hand to hold/operate a syringe and is intended to increase rates at which fluids may be manually forced through injection elements (e.g. needle). U.S. Pat. No. 8,539,644 dated Sep. 24, 2013, entitled Hinge Assembly for an Injector by Shawn P. Fojtik, the disclosure of which is incorporated herein by reference, is a related invention that provides an injector with a forward and rear handle member connected at a hinge. The hinge defines an axis of rotation such that the rotation can be prevented if the mechanism is in the locked configuration.

United States Patent Application Publication Number US 2014/0066862 dated Mar. 6, 2014 entitled Ergonomic Syringe Systems by Dennis Schweers, the disclosure of which is incorporated herein by reference, describes an ergonomic syringe system that provides attachments for dispensing or drawing in fluid to be used for the increased comfort and reduced risk of repetitive strain injuries. One such embodiment provides a grip that may be mounted to the barrel and/or plunger of a syringe to provide a more ergonomic way to use the syringe. U.S. Pat. No. 8,647,115 dated Feb. 11, 2014 entitled Syringes for Dispensing Multi-Component Material by Andreas J. Boehm et al., the disclosure of which is incorporated herein by reference, provides an invention with a similar design to the previously discussed patents but with a different application of the design. This invention relates to syringes for dispensing multi-component materials and includes a syringe cartridge in an applicator used to fill the syringe cartridge.

### SUMMARY OF THE INVENTION

A reciprocating syringe injector includes an inside arm and an outside arm pivotally mounted to the inside arm. A spring bias biases the inside arm away from the outside arm. A small syringe retainer is formed on the inside arm. A ratchet member extends from the inside arm toward the outside arm. The ratchet member has ratchet teeth protruding from the ratchet member. A ratchet engagement extension is pivotally mounted to the outside arm. The ratchet engagement extension has a ratchet engagement edge that engages the ratchet teeth formed on the ratchet member.

The reciprocating syringe injector also optionally includes a large reservoir retainer mounted to the small syringe retainer. The large reservoir retainer could also be mounted anywhere on the inside arm. A ratchet release trigger is pivotally mounted to the outside arm. The ratchet release trigger is connected to the ratchet engagement extension and configured so that rotation of the ratchet release trigger rotates the ratchet engagement extension away from the ratchet teeth to disengage the ratchet engagement edge from the ratchet teeth.

A finger guard is mounted on a lower edge of the inside arm and an inside arm grip includes an index finger indentation formed under the finger guard, a middle finger indentation, a ring finger indentation, and a little finger indentation. An inside arm syringe channel is formed on the small syringe retainer and a small syringe lower slot retainer is formed as a horizontal slot normal to the inside arm syringe channel. The small syringe lower slot retainer is formed underneath a small syringe lower protrusion retainer. An outside arm upper retaining abutment extends from the outside arm. The outside arm retaining abutment is linearly disposed over the small syringe retainer. he outside arm upper retaining abutment is pivotally connected to the outside arm. An outside arm lower prong opposes the outside arm upper retaining abutment with an outside arm slot formed as a gap between the outside arm lower prong and the outside arm upper retaining abutment.

### BRIEF DESCRIPTION OF THE DRAWINGS

FIG. 1 is a perspective view of the reciprocating syringe injector.

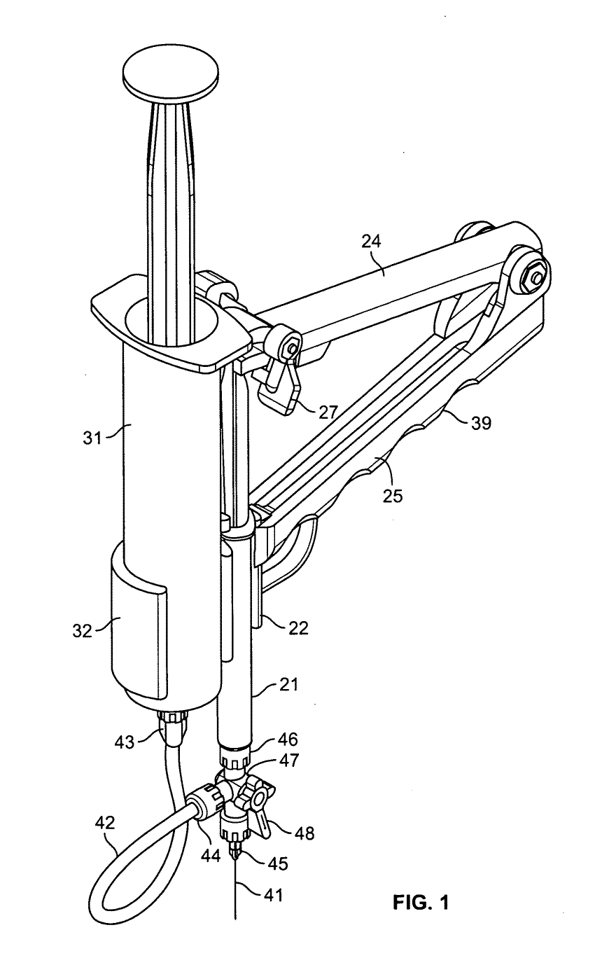

FIG. 2 is an exploded perspective view of the reciprocating syringe injector.

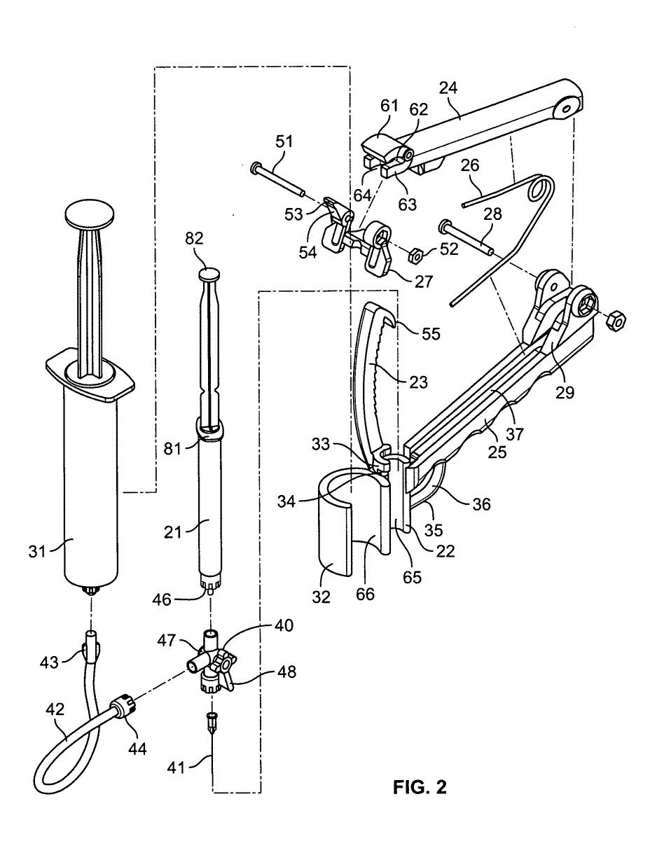

FIG. 3 is a right side view of the reciprocating syringe injector.

FIG. 4 is a left side view of the reciprocating syringe injector.

The following call out list of elements can be a useful guide in referencing the element numbers of the drawings.
21 Small Syringe
22 Small Syringe Retainer
23 Ratchet Member
24 Outside Arm
25 Inside Arm
26 Torsion Spring
27 Ratchet Release
28 Ratchet Axle
29 Ratchet Hinge
31 Large Reservoir
32 Large Reservoir Retainer
33 Small Syringe Lower Protrusion Retainer
34 Small Syringe Lower Slot Retainer
35 Finger Guard
37 Torsion Spring Slot
39 Inside Arm Grip
40 Valve
41 Needle
42 Catheter
43 First Catheter Connector
44 Second Catheter Connector
45 Needle Connector
46 Valve Body Connector
47 Valve Body
48 Valve Arm
51 Ratchet Release Bolt
52 Ratchet Release Nut
53 Ratchet Engagement Edge
54 Ratchet Engagement Extension
55 Ratchet Upper Stop
56 Ratchet Tooth
57 Outside Arm Spring Retainer
61 Outside Arm Upper Retaining Abutment
62 Outside Arm Slot
63 Outside Arm Lower Prong
64 Outside Arm Syringe Slot
65 Inside Arm Syringe Channel
66 Inside Arm Reservoir Channel
67 Needle Connector Backflow Valve
68 Catheter Connector Backflow Valve
71 Index Finger Indentation
72 Middle Finger Indentation
73 Ring Finger Indentation
74 Little Finger Indentation
75 Magnetic Base
81 Small Syringe Forward Handle
82 Small Syringe Extended Handle

### DETAILED DESCRIPTION OF THE PREFERRED EMBODIMENT

The present invention is a reciprocating syringe used for injecting liquid into a patient. A variety of liquids can be injected into the patient including a variety of drugs. The invention includes a frame that is handheld and is designed to reciprocate to provide a continuous injection into a patient without stopping and withdrawing the needle from the patient for reloading. The reciprocating syringe frame draws liquid from a large reservoir 31 into a small syringe 21 and then injects liquid from the small syringe 21. Then the large reservoir 31 refills the small syringe 21.

As seen in the figures, and inside arm 25 opposes an outside arm 24. The inside arm has an inside arm grip 39 with finger indentations including an index finger indentation 71, a middle finger indentation 72, a ring finger indentation 73, and a little finger indentation 74. The finger indentations receive a person's hand and allow a stabilized grip. The reciprocating syringe injector injects when a user squeezes the finger indentations against the outside arm 24. The outside arm 24 is smooth and rounded to receive a palm of a hand. A finger guard 35 can further enhance fine positioning of the syringe injector and can help with stabilization during reloading. Optionally, a magnetic base of 75 can retain the injector so that the injector can magnetically adhere to a flat metal surface, or can retain ferrous articles.

The reciprocating syringe frame is controlled by a ratchet that controls motion of the outside arm 24 which rotates relative to the inside arm 25. The outside arm 24 is pivotally mounted to the inside arm 25. The outside arm 24 is biased away from the inside arm 25 by a torsion spring 26. The torsion spring has a pair of extensions that are biased away from each other and are both connected at a coil which can have two turns or more. The torsion spring upper extension fits into the outside arm 24 and the torsion spring lower extension fits into the inside arm 25. The torsion spring slot 37 receives the torsion spring lower extension when the torsion spring lower extension is linearly seated into the torsion spring slot 37. The torsion spring slot 37 can have a snap fit mechanical connection with the torsion spring 26. Similarly, the upper extension of the torsion spring 26 can fit into an outside arm spring retainer 57. The outside arm spring retainer 57 can also be a snap fit, or a channel that encapsulates at least a portion of the torsion spring that is engaged and fitted to the outside arm. The coil of the torsion spring can fit through the axis of rotation of the outside arm and inside arm, or can be outside of the axis of rotation.

A ratchet axle 28 can define the axis of rotation of the outside arm in the inside arm. The ratchet axle 28 can be formed as a pin that can be a bolt which engages a nut such as a ratchet axle nut. The ratchet axle nut can be captured within a hexagonal shaped thermoplastically formed socket that is formed on the ratchet hinge 29 of the inside arm 25. The ratchet hinge 29 therefore comprises the ratchet axle 28 and the ratchet axle nut 29 as well as a torsion spring 26 which biases the outside arm 24 away from the inside arm 25.

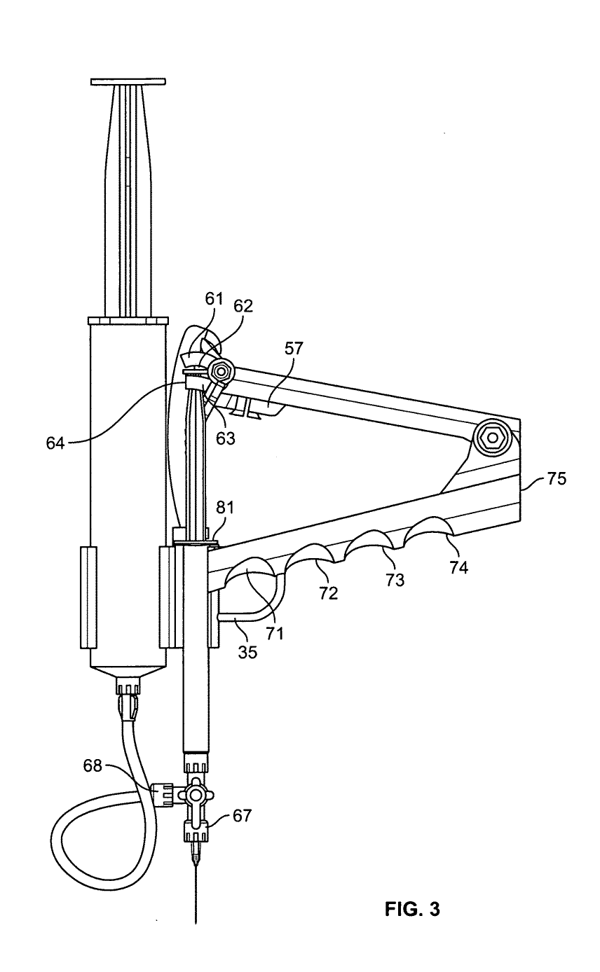

The ratchet engagement extension 54 can be rotationally biased toward the ratchet member 23. For example, a spring can be used such as a rubber band. A rubber band or other type of spring can fit around a rubber band spool, as seen in FIG. 3, near or on the outside arm spring retainer 57. The rubber band spool has a gap that defines a right side and left side. The right side and left side can rotate away from each other which activates a spring bias of the rubber band. Thus, the outside arm spring retainer 57 can retain both the torsion spring 26 and the rubber band.

Preferably, a small torsion spring such as a ratchet release bolt torsion spring can be mounted to the ratchet release bolt 51 so that the torsion spring biases the ratchet engagement edge 53 and the ratchet engagement extension 54 rotationally toward the ratchet member 23. The coil of the ratchet release bolt torsion spring can be whipped about the shaft of the ratchet release bolt between the ratchet engagement extension 54 and the ratchet release 27. The ratchet release bolt passes 51 through the ratchet engagement extension 54 at a ratchet engagement extension opening and the ratchet release bolt 51 also passes through the ratchet release 27 at a ratchet release opening. The ratchet engagement extension opening and the ratchet release opening received the ratchet release bolt 51 in a rotational configuration through a ratchet release passage formed on the outside arm 24. The ratchet release passage formed on the outside arm 24 can have a clearance for receiving a coil portion of the torsion spring that rotationally biases the ratchet engagement extension 54 toward the ratchet member 23.

A ratchet member 23 has an arc-shaped profile defined by a circumscribed portion of a ratchet engagement edge 53 which engages individual ratchet teeth 56 formed as extensions on the ratchet member 23. The individual ratchet teeth 56 extend toward the ratchet engagement extension 54 and also toward the ratchet axle 28. The ratchet engagement extension 24 extends towards the ratchet member 23 which extends outwardly and upwardly on a right side of the inside arm. As a user depresses the ratchet engagement edge 53 against the ratchet member 23, the ratchet teeth 56 can provide a clicking sound for giving a user audible feedback. Each ratchet tooth can correspond with or be calibrated with a particular dosage for example.

The ratchet engagement edge 53 is formed on a ratchet engagement extension 54 that is pivotally mounted to the outside arm 24. A ratchet release trigger 27 is also pivotally mounted to the outside arm 24. The user can activate the ratchet release trigger with a thumb while the user has a grip on the outside arm and the inside arm. The ratchet release trigger is connected to the ratchet engagement extension 54 and the ratchet engagement edge 53. When the ratchet release trigger 27 is rotated toward the ratchet member 23, the ratchet engagement edge 53 disengages from the ratchet teeth 56 of the ratchet member 23. Disengaging the engagement edge 53 allows the torsion spring 26 to open the outside arm 24 away from the inside arm 25.

The inside arm 25 is shaped to receive a syringe such as a small syringe 21. The small syringe 21 fits inside the small syringe retainer 22. The small syringe retainer 22 can be integrally formed by metal or injection molded plastic with the inside arm 25. A large reservoir 31 can be mounted with the small syringe 21. The large reservoir 31 can be mounted to a large reservoir retainer 32. The large reservoir retainer 32 can be connected to the small syringe retainer 22 and can be integrally molded with the small syringe retainer 22. Additionally, the small syringe retainer 22 can have an inside arm syringe channel 65 that cradles a cylindrical sidewall of the small syringe 21. Similarly, the large reservoir retainer 32 can have an inside arm reservoir channel 66 that cradles a cylindrical sidewall of the large reservoir 31 if the large reservoir 31 is formed with a cylindrical sidewall. The large reservoir 31 can be formed as a large syringe that is larger than the small syringe.

The inside arm syringe channel 65 begins at a level close to the torsion spring slot, and terminates at a level below the finger guard 35. The inside arm syringe channel 65 can be cylindrically formed and have an opening facing a right side of the inside arm 25. Similarly, the inside arm reservoir channel 66 can also be cylindrically formed and have an opening facing the right side of the inside arm 25. If the resulting pair of openings are rectangular in shape, then the small syringe retainer 22 and the large reservoir retainer 32 can both be formed as clips that clip onto the various container dispensers of liquid.

The small syringe has a forward handle called a small syringe forward handle 81 that is typically engaged against an index finger and middle finger. The small syringe also has an extended handle 82 typically engaged against a thumb. The extended handle 82 is typically depressed and travels toward the needle while dispensing liquid. The extended handle of the small syringe is retained to the outside arm 24. The outside arm 24 has an outside arm upper retaining abutment 61 that acts as a thumb for pressing on the syringe extended handle. The syringe extended handle can be formed as a disk that fits into an outside arm slot formed on an end of the outside arm 24. The outside arm upper retaining abutment 61 can have a smooth protrusion for allowing a user to rest a thumb on the outside arm upper retaining abutment 61. Additionally, the outside arm upper retaining abutment 61 can be pivotally mounted to the outside arm 24 so that the outside arm slot 62 remains parallel with the forward handle of the small syringe 21. The small syringe 21 can be retracted for aspirating liquid when the outside arm syringe slot 64 fits over the small syringe to allow an outside arm lower prong 63 to pull the small syringe extended handle upward to draw more liquid. The liquid is drawn from the reservoir 31 which can be formed as a larger syringe.

The outside arm syringe slot 64 is vertically oriented to receive a post of the small syringe 21. The outside arm slot 62 is horizontally oriented for receiving a extended handle 82 of the small syringe 21. The horizontal and vertical, lower and upper positions are in reference to the drawings as shown in the figures. In actual use, the syringe injector can be used diagonally, upside down or in the regular orientation as shown in the figures.

The small syringe forward handle 81 is typically formed as a flange that is plastic injection molded with the main body of the small syringe 21. The small syringe extended handle 82 is typically formed as a flat plastic disk mounted on a crossbeam extension post extending rearwardly from the small syringe 21. When the flange is received into the small syringe lower slot retainer 34, it preferably fits under the small syringe lower protrusion retainer 33 which can have a grippy surface for snapping to, or otherwise fitting to the small syringe 21. The other end of the flange fits over a portion of the inside arm. The small syringe forward handle can be formed as a flat plastic member that fits into a small syringe lower slot retainer 34. The small syringe lower slot retainer is formed between the small syringe lower protrusion retainer 33 and an upper surface of the inside arm syringe channel 65.

A user first loads a small syringe into the syringe injector device. The small syringe can snap onto the inside arm syringe channel 65 in a releasable connection, with the small syringe forward handle 81 lodged to the inside arm 25 at the small syringe lower slot retainer 34. Secondly, the user can attach a reservoir 31 as a larger syringe. The reservoir snaps to the inside arm reservoir channel 66. The user can then connect a valve 40 with the small syringe and to the large syringe. A needle 41 is connected to the valve 40 at a lower opening of the valve 40. A plastic flexible tubular member such as a catheter 42 can connect to the reservoir 31 and the valve 40. The catheter 42 has a first catheter connector 43 that connects to the large reservoir 31. The catheter 42 has a second catheter connector 44 that connects to a side opening of the valve 40. The needle connector 45 connects the needle 41 to the lower opening of the valve 40. The valve body connector 46 connects the upper opening of the valve body 47 to the small syringe 21. The valve body 47 has three openings, namely an upper opening connected to the small syringe, a lower opening connected to the needle 41 and a side opening connected to the large reservoir 31 through the catheter 42.

The valve can have a selectable function. For example, a valve arm 48 can be mounted to the valve body 47 so that the valve arm 48 rotates in a 90° quarter turn or in a 180° half turn for selecting a variety of different functions. The valve arm 48 can provide a flow-through mode where the small syringe 21 injects straight to the needle 41 or a stop mode where the valve body does not allow fluid to pass through it. Typically, a user can inject the contents of the small syringe 21 in small injections, and then when the small syringe is exhausted, the user can rotate the valve arm 48 so that the small syringe 21 is in fluid communication with the large reservoir 31 through the catheter 42. The user then draws liquid from the large reservoir 31 into the small syringe 21 by depressing the ratchet release trigger 27. The ratchet release trigger 27 rotates the ratchet engagement edge 53 to release the ratchet engagement edge 53 from the ratchet member 23 which in turn allows the torsion spring 26 to bias the outside arm 24 away from the inside arm 25. The bias of the outside arm 24 away from the inside arm raises the outside arm lower prong 63. Because the outside arm syringe slot 64 holds the small syringe extended handle 82, the pair of outside arm lower prongs 63 bias the small syringe 21 into an aspiration motion for receiving more liquid from the large reservoir 21.

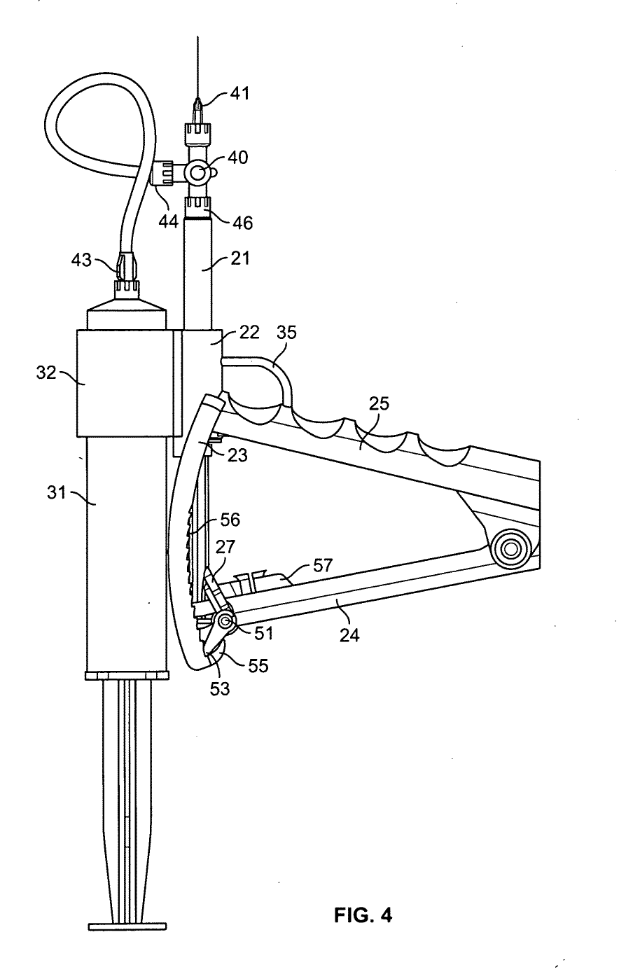

When the small syringe is filling, the ratchet engagement edge 53 does not inhibit motion since it is disengaged. The ratchet engagement edge 53 rotates relative to the ratchet release bolt 51. The ratchet engagement extension 54 is rotationally mounted and pivotally mounted to the ratchet release bolt 51. The ratchet release bolt 51 preferably has a mostly smooth bore with a threaded tip that engages the ratchet release nut 52. The ratchet release nut 52 fits within a hexagonal socket formed on the ratchet release trigger. The ratchet engagement extension 54 eventually bottoms out and abuts the ratchet upper stop 55. Then the ratchet engagement edge 53 sets into the outermost ratchet notch below the outermost ratchet tooth when the ratchet upper stop 55 rotationally biases the ratchet engagement extension 54 clockwise as seen in FIG. 4. The user can then manually rotate the valve arm 48 and resume injecting through the needle 41 again.

To avoid the requirement for manually rotating the valve arm 48 every time the user needs to change between injection and aspiration motions of the small syringe, a pair of back flow valves can be installed to allow continuous reciprocating injection of the small syringe 21. For example, a needle connector backflow valve 67 can prevent fluids from a patient to enter the needle. Also, a catheter connection backflow valve 68 can prevent fluids from entering the catheter and traveling to the reservoir. Thus, with the pair of back flow valves, also called one-way valves, the device can operate in a semi automatic reloading fashion. Now the user does not need to rotate the valve arm 48 for manually selecting the flow path through the valve 40. The valve arm 48 can be used only for shutting off the flow to the needle or reservoir in case of needle or reservoir changes. Either the manual or semi automatic method can be used. The back flow valves can be formed as ball valves or as flap valves. No best mode exists as between the manual or semi automatic configuration and the configuration can be changed in mode in the field using commonly and commercially available components.

A variety of modifications can be made to the present invention without departing from the scope of the claims as defined below. For example, the outside arm upper retaining abutment 61 can have a thumb grip for more comfortable manual positioning. The outside arm slot 62 can be single-sided rather than double-sided as shown in the figures. The outside arm lower prong 63 can be integrally formed with the outside arm upper retaining abutment 61. The ratchet member 23 and ratchet engagement edge 53 can be formed with a smooth arm and an edge that engages via friction. The torsion spring 26 can be replaced with a rubber spring, an electric motor, or two opposing magnets.

1. A reciprocating syringe injector comprising:
an inside arm;
an outside arm pivotally mounted to the inside arm;
a spring bias biasing the inside arm away from the outside arm;
a small syringe retainer formed on the inside arm;
a ratchet member extending from the inside arm toward the outside arm, where the ratchet member has ratchet teeth protruding from the ratchet member;
a ratchet engagement extension pivotally mounted to the outside arm, were in the ratchet engagement extension has a ratchet engagement edge that engages the ratchet teeth formed on the ratchet member.

2. The reciprocating syringe injector of claim 1, further including: a large reservoir retainer mounted to the small syringe retainer.

3. The reciprocating syringe injector of claim 1, further including: a large reservoir retainer mounted to the inside arm.

4. The reciprocating syringe injector of claim 1, further including: a ratchet release trigger pivotally mounted to the outside arm, wherein the ratchet release trigger is connected to the ratchet engagement extension and configured so that rotation of the ratchet release trigger rotates the ratchet engagement extension away from the ratchet teeth to disengage the ratchet engagement edge from the ratchet teeth.

5. The reciprocating syringe injector of claim 1, further including: a finger guard mounted on a lower edge of the inside arm and an inside arm grip which includes an index finger indentation formed under the finger guard, a middle finger indentation, a ring finger indentation, and a little finger indentation.

6. The reciprocating syringe injector of claim 1, further including: an inside arm syringe channel formed on the small syringe retainer and; a small syringe lower slot retainer formed as a horizontal slot normal to the inside arm syringe channel, were in the small syringe lower slot retainer is formed underneath a small syringe lower protrusion retainer.

7. The reciprocating syringe injector of claim 1, further including: an outside arm upper retaining abutment extending from the outside arm, wherein the outside arm retaining abutment is linearly disposed over the small syringe retainer, wherein the outside arm upper retaining abutment is pivotally connected to the outside arm.

8. The reciprocating syringe injector of claim 7, further including: an outside arm lower prong opposing the outside arm upper retaining abutment with an outside arm slot formed as a gap between the outside arm lower prong and the outside arm upper retaining abutment.

9. The reciprocating syringe injector of claim 8, further including: a large reservoir retainer mounted to the small syringe retainer.

10. The reciprocating syringe injector of claim 8, further including: a large reservoir retainer mounted to the inside arm.

11. The reciprocating syringe injector of claim 8, further including: a ratchet release trigger pivotally mounted to the outside arm, wherein the ratchet release trigger is connected to the ratchet engagement extension and configured so that rotation of the ratchet release trigger rotates the ratchet engagement extension away from the ratchet teeth to disengage the ratchet engagement edge from the ratchet teeth.

12. The reciprocating syringe injector of claim 8, further including: a finger guard mounted on a lower edge of the inside arm and an inside arm grip which includes an index finger indentation formed under the finger guard, a middle finger indentation, a ring finger indentation, and a little finger indentation.

13. The reciprocating syringe injector of claim 8, further including: an inside arm syringe channel formed on the small syringe retainer and; a small syringe lower slot retainer formed as a horizontal slot normal to the inside arm syringe channel, were in the small syringe lower slot retainer is formed underneath a small syringe lower protrusion retainer.
